(19) United States
(12) Patent Application Publication
(10) Pub. No.: US 2008/0118542 A1
(43) Pub. Date: May 22, 2008

(54) Growth Factor Composition

(75) Inventors: Claes Arnander, Nacka (SE); Thomas Engstrand, Uppsala (SE); Olle Larm, Bromma (SE); Riikka Veltheim, Stockholm (SE)

Correspondence Address: BIRCH STEWART KOLASCH & BIRCH, PO BOX 747, FALLS CHURCH, VA, 22040-0747, US

(73) Assignee: BONOSS MEDICAL AB, STOCKHOLM SWEDED (SE)

(21) Appl. No.: 11/795,234

(22) PCT Filed: Jan. 19, 2006
(86) PCT No.: PCT/SE06/00077

§ 371 Date: Sep. 12, 2007

(30) Foreign Application Priority Data
Jan. 19, 2005 (SE) 0500132-6

(51) Int. Cl. A61K 38/18; A61K 47/36; A61L 27/26; A61P 19/00

(52) U.S. Cl. 424/423; 514/777; 514/12

(57) ABSTRACT

A composition is provided, which is capable of generating hard tissue when introduced in a mammalian subject and comprises a) an ionic complex of i) chitosan and ii) a negatively charged polysaccharide selected from the group consisting of heparin, heparan sulfate and dextran sulfate, and b) a hard tissue generating growth factor, said ionic complex being a carrier for said hard tissue generating growth factor.

Also provided are a method of preparation in vitro of a bone graft substitute using the composition, a kit for carrying out the method, as well as use of the composition in the preparation of a medical device for generation of hard tissue in a mammalian subject in need thereof, and a method of generation of hard tissue at a desired site in a mammalian subject in need thereof.

### FIELD OF THE INVENTION

The present invention relates to the field of growth factor compositions, and in particular to the provision of a hard tissue generating growth factor in a suitable carrier. The present invention furthermore concerns the field of hard tissue reconstruction and manufacture of implants, e.g. bone graft substitutes.

### BACKGROUND

### Growth Factors

Growth factors are proteins that bind to receptors in the glycocalyx on the cell surface, with the primary result of activating cellular proliferation and/or differentiation. Many growth factors are quite versatile, stimulating cellular division in numerous different cell types, while others are specific to a particular cell type. Heparan sulfates are the most well-known and thoroughly studied receptors for growth factors. Heparan sulfates are present on the surface of almost all mammalian cells (examples are syndecan, perlecan, glypican). The carbohydrate portions of these heparan sulfates are highly charged (sulfated), heterogeneous polysaccharides. The polysaccharides (glycosaminoglycans or GAGs) are responsible for the specific biological activities of the glycoconjugate and are inter alia utilized by the growth factors for binding.

Examples of growth factors include bone morphogenetic proteins (BMPs). BMPs are secreted proteins that belong to the TGF-β superfamily. BMPs were originally identified and purified from demineralized bone, and were characterized by their ability to induce bone formation at ectopic sites in muscle. Molecular cloning of BMP-2 and BMP-4 confirmed the bone-inductive capacity of these molecules. Most BMPs are found in bone and are essential mediators of osteogenic cell commitment and differentiation. In several in vitro models, BMPs have been shown to initiate the differentiation of mesenchymal cells into the osteogenic lineage and induce expression of osteocalcin and alkaline phosphatase, two bone-associated markers. Several in vitro and in vivo studies have demonstrated the osteoinductive capacity of recombinant human (rh) BMP-2, -4, -6, -7, -9, and -14.

### Carriers for Hard Tissue Generating Growth Factors

Similarly to the effect of many growth factors, the osteoinductive effect of different BMPs is highly dependent on the delivery vehicle. BMPs are water-soluble dimeric proteins that diffuse easily in the body fluids. When administered locally in vivo, without a carrier, they do not endure more than minutes at the deposited site. The need for a carrier has been recognized since the possible clinical use of BMPs was identified, and various carriers have been investigated, both experimentally and clinically (as reviewed in e.g. “Polymeric growth factor delivery strategies for tissue engineering”, Chen R R and Mooney D J, Pharm Res 20(8):1103-12 (2003)). BMP carriers can be broadly classified into inorganic salts, naturally occurring polymeric substances, synthetic polymers, and composites of synthetic and naturally occurring polymers. An ideal carrier should neither induce an inflammatory response nor cause an immune reaction. Degradation of the carrier should not result in toxic residues. Ideally, the carrier should be absorbed concurrently with bone healing, leaving no residue. At present, collagen is the most commonly used carrier for BMPs, and type 1 collagen is preferred. Type 1 collagen can be obtained from skin, bone, tendons and ligaments. Bovine collagen is currently used as carrier for many growth factors in the clinical setting, and has been approved by FDA for human use. Because BMPs can be easily squeezed out of the collagen by axial loads and under pressure, it has to be contained in a cage for interbody fusion. Products comprising osteogenic protein-1 (OP-1, also referred to as BMP-7) employ bone-derived collagen as carrier. This collagen binds strongly to the BMP, presumably through hydrogen bonding. Demineralized bone matrix as a carrier for BMPs has not gained popularity because of the risk of immunogenicity and the risk of disease transmission. Other natural polymers that have been considered as carriers for growth factors are hyaluronic acid, fibrin, chitosan, alginate, and other animal- or plant-derived polysaccharides. None of these has yet gained acceptance for use in humans. Synthetic polymers, which is another category of suggested carrier materials for growth factors, carry the advantages of unlimited supply, low or no antigenicity, predictable degradation products, and no risk of disease transmission. Although synthetic polymers such as polyglycolic acid and polylactic acid derivatives have been explored, their degradation products can produce giant cell reaction and the binding affinity of BMPs to these synthetic polymers is not good.

### Stabilization and Potentiation of Growth Factors

It is known that heparin, a glycosaminoglycan which is structurally related to heparan sulfate, stabilizes and potentiates the functional activity of a large number of growth factors, including the BMPs. This is probably due to the fact that, as is mentioned above, these growth factors utilize heparin sulfates as receptors. Binding of protein to heparin, as well as to heparan sulfate, is highly specific and requires unique sequences of monosaccharide units that are present in both polysaccharides.

The possibility of using heparin as a stabilizer and activator for BMPs has been investigated, but the attempts have enjoyed limited success. The reasons for the limited success are the occurrence of bleeding complications and the short durability of heparin in vivo. On average, heparin has a half-life in blood and other tissues of less than 90 minutes.

### Heparin

Heparin is a commercially available polysaccharide, which is isolated from mammalian tissue (pig mucosa or beef lung). Since its discovery in 1916 by Jay McLean, heparin has been recognized for its blood anticoagulant properties. Heparin has been used clinically for more than 50 years as a blood anticoagulant and antithrombotic agent. In contrast to heparan sulfates, heparin is present only in the basophilic granules of mast cells. Today, it is extensively used in the clinic as a blood anticoagulant and/or anti-inflammatory agent.

Heparin and heparan sulfate glycosaminoglycans are built up by alternating D-glucosamine and uronic acid residues (L-iduronic and D-glucuronic). They are highly charged (sulfated) heterogeneous polysaccharides.

### Chitosan

The polysaccharide chitin is the second most abundant organic compound in nature after cellulose. Commercially, chitin is obtained mainly from crab and shrimp shells. Chitin has a regular structure and is composed of β-1,4-linked N-acetyl-D-glucosamine residues. Chitosan is positively charged and is obtained by partial N-deacetylation of chitin, e.g. via treatment of chitin with a strong base.

In vivo, chitosan is degraded by lysozyme and other glycosaminodases to mono- and oligomers. A chitosan which is rich in N-acetyl-D-glucosamine residues is degraded faster in vitro, and probably also in vitro, than a chitosan with a high proportion of D-glucosamine residues.

Chitosan has been suggested as a delivery vehicle for growth factors (see e.g. U.S. Pat. No. 6,124,273 discussed below). However, there are concerns since chitosan has been shown to initiate blood clotting. Chitosan has been approved by the US FDA as a first-aid bandage to reduce haemorrhage in traumatic wounds. The use of chitosan alone as a vehicle for growth factors may therefore be limited, since chitosan potentially can induce thrombosis and necrotic cell death at the implantation site.

### PRIOR ART

U.S. Pat. No. 5,894,070 describes the use of an agent comprising chitosan and a polysaccharide immobilized thereto for stimulation of regeneration of hard tissue. The polysaccharide is selected from heparin, heparan sulfate, chondroitin sulfates and dextran sulfate. The experiments described in this document enable use of a chitosan-heparin composition lacking any additional components for stimulation of bone regeneration.

U.S. Pat. No. 6,124,273 describes a system for sustained release of active substances in vivo, using e.g. chitosan in hydrogel form as a carrier for delivery of a growth factor.

WPI abstract no. 2003-753295 of Korean publication KR2003011407 describes a composition comprising a gel forming agent, e.g. chitosan; a bone morphogenetic protein; an antibiotic; and a pain-relieving agent. The composition is intended for use as a dentine forming agent.

US patent application publication 2003/0158302 discloses self-forming mineral-polymer hybrid compositions comprising a liquid component and a solid component. The liquid component may comprise chitosan as a sub-component. The composition may furthermore comprise growth factors, e.g. bone morphogenetic proteins.

U.S. Pat. Nos. 6,773,723 and 6,936,276 describe biodegradable matrices comprising two polymeric layers, useful for tissue regeneration. Drugs, growth factors, polypeptides, proteins, cDNA, gene constructs and other bioactive therapeutic agents may be included in the matrix. The bilayer matrix comprises at least two porous polymeric layers that differ in their composition, density and porosity, so that they have different characteristics within the environment of growing tissue. The two polymeric layers are prepared separately from each other. Examples of polymers for use in forming the separate layers, mention is made of many different polymers, e.g. chitosan and heparin. There is no mention of an ionic complex of chitosan and heparin.

### DISCLOSURE OF THE INVENTION

The present invention has as its main object the provision of a new and efficient technique for the delivery of hard tissue generating growth factors to a subject in need thereof, for example for the purpose of generation of hard tissue.

A second object of the invention is to provide a formulation for delivery of a hard tissue generating growth factor, which formulation acts to stabilize the hard tissue generating growth factor and promote its effect in a clinical situation or otherwise, for example the effect of generation of new bone tissue.

A third objective of the invention is to provide a formulation comprising a hard tissue generating growth factor, which is possible to design such that it substantially remains at the site of treatment when administrated.

A fourth objective of the invention is to create a hard tissue generating growth factor formulation, which is possible to shape in such a way that it mimics the shape of the hard tissue that is to be generated in the region of its administration.

A fifth objective of the invention is to provide methods for in vivo and in vitro generation of hard tissue, for example bone or cartilage.

For these and other objects the invention, in a first aspect, provides a composition capable of generating hard tissue when introduced in a mammalian subject, the composition comprising:
a) an ionic complex of i) chitosan and ii) a negatively charged polysaccharide selected from the group consisting of heparin, heparan sulfate and dextran sulfate, and
b) a hard tissue generating growth factor, said ionic complex being a carrier for said hard tissue generating growth factor.

Thus, it has been found by the present inventors that an ionic complex of chitosan and a negatively charged polysaccharide can be advantageously used as a carrier for a growth factor. When solutions of negatively charged polysaccharide and chitosan are mixed, an ionic complex is immediately formed and precipitates. Surprisingly, it was found that chitosan protects the negatively charged polysaccharide from enzymatic degradation in vivo, and that the half-life of the negatively charged polysaccharide is thereby considerably prolonged. Unexpectedly, growth factors are stabilized over a longer period when included in the ionic complex of chitosan and negatively charged polysaccharide.

As mentioned in the background section, the negatively charged polysaccharide heparin may, even at moderate concentrations, cause bleeding complications when introduced by itself into mammalian tissue. In an ionic complex with chitosan, heparin is tightly linked to chitosan by ion bonds, which means that the risk for bleeding complications is substantially reduced. As mentioned, chitosan can induce thrombosis and necrotic cell death when implanted. However, the combination of negatively charged polysaccharide and chitosan has been demonstrated to inhibit platelet adhesion and activation. The ionic complex of chitosan and negatively charged polysaccharide has therefore been found to be advantageous as an implantable or injectable carrier or matrix, which reduces the risk for thrombosis.

In summary, some of the advantages of the inventive ionic complex of chitosan and negatively charged polysaccharide as a carrier for a growth factor are:
the negatively charged polysaccharide is stabilized by chitosan in the ionic complex and enzymatic degradation thereof is substantially delayed;
the negatively charged polysaccharide reduces or eliminates the risk of blood clotting effects of chitosan;
chitosan reduces or eliminates the risk of bleeding complications as it deactivates the blood anticoagulant properties of the negatively charged polysaccharide.

In a preferred embodiment of the composition according to this aspect of the invention, the number of positive charges contributed by said chitosan are in excess over the number of negative charges contributed by said negatively charged polysaccharide in the ionic complex. Upon administration of this embodiment of the inventive composition, with an excess of chitosan in comparison to negatively charged polysaccharide on a charge basis, the composition is immobilized at the site of treatment. This is because in general, all cells are negatively charged. The immobilization of the composition in the area to be treated results in a gradual and local release of growth factor and negatively charged polysaccharide will be obtained.

Non-limiting examples of the hard tissue generating growth factor in the composition according to the invention are selected from the group consisting of BMP-2, BMP-4, BMP-6, BMP-7, BMP-9 and BMP-14. Among these, a preferred growth factor is BMP-2.

In a preferred embodiment of the inventive composition, the negatively charged polysaccharide is heparin. Thus, in this embodiment, the inventive composition consists of a chitosan-heparin ionic complex carrying a growth factor. In such an ionic complex, the weight ratio chitosan:heparin may be from about 1:2 to 10:1, such as from about 1:1 to about 5:1, for example from about 2:1 to about 5:1. Examples of more specific intervals are from about 3:1 to about 4:1, and from about 2:1 to about 3:1. The weight ratio of chitosan:heparin in the ionic complex affects the physical characteristics of the complex, in particular its rheological properties and adhesiveness. Furthermore, having an excess of heparin of more than double the amount of chitosan would entail a substantial risk of unwanted anticoagulation effects from the heparin. The ranges given above are to be seen as guidelines for the skilled person to find the optimal ratio based on the particular situation in which the composition is to be used.

In embodiments of the inventive composition, the chitosan in the ionic complex carrier may have a degree of deacetylation of from about 50% to about 98%, such as from about 50% to about 95%, for example from about 80% to about 90%. The degree of deacetylation affects the solubility of chitosan and its rate of degradation. The above preferred ranges are guidelines for the skilled person to find the optimal deacetylation degree based on the particular situation in which the composition is to be used.

What concentration of growth factor to employ in the composition according to the invention may vary within wide limits. For example, the content of said hard tissue generating growth factor may be from about 0.1 to about 10 percent by weight, preferably from about 0.5 to about 5 percent by weight, based on the total weight of ionic complex and hard tissue generating growth factor.

The ionic complex of chitosan and negatively charged polysaccharide may be formulated into any physical form. In a preferred embodiment, the complex is in the form of a gel. In case of a complex in the form of a gel, the growth factor may be present in a concentration of from about 5 to about 500 μg/ml gel, preferably from about 1 to about 100 μg/ml gel.

In an even more preferred embodiment, the complex is in the form of a lyophilizate. In case of a complex in the form of a lyophilizate, the hard tissue generating growth factor may be present in a concentration of from about 1 to about 50 μg/mg lyophilizate, preferably from about 2 to about 25 μg/mg lyophilizate. An ionic complex of chitosan and negatively charged polysaccharide in lyophilized form has a dry, sponge-like appearance. Shaping of the complex, with or without growth factor, prior to lyophilization enables preparation of such sponge-like lyophilizates of any desired shape. If not added previously, a solution of hard tissue generating growth factor can be adsorbed onto the lyophilizate in order to provide the inventive composition.

Surprisingly, a composition according to this embodiment of the invention, i.e. a composition comprising a growth factor in a lyophilizate carrier, for example in the form of a sponge-like structure, exhibits an effect which is more beneficial than the effect of other physical forms of the inventive composition. The surprising influence of physical form on growth factor effect is demonstrated for the growth factor BMP-2 in the examples that follow. It is believed that the advantages of using the ionic complex in lyophilized form are due to the fact that hard tissue formation is defined in terms of size and form by the implanted “sponge”. Using a carrier of this type avoids uncontrolled spreading of bone formation, which is of critical importance in many applications (see e.g. Poynton A R and Lane J M, Spine 15;27 (16 Suppl 1):S40-8 (2002)).

In further aspects of the invention, the advantageous properties of a composition according to the first aspect of the invention are applied to the field of bone reconstruction.

Thus, a second aspect of the invention provides a method of preparation in vitro of a bone graft substitute, which method comprises:
a) providing an ionic complex of i) chitosan and ii) a negatively charged polysaccharide selected from the group consisting of heparin, heparan sulfate and dextran sulfate,
b) shaping said ionic complex in a desired shape of a bone graft substitute, and
c) allowing said ionic complex to set into a solid or semi-solid bone graft substitute structure with said desired shape,
   which method also comprises the step of adding a hard tissue generating growth factor to said ionic complex.

The hard tissue generating growth factor may be added to the complex at any stage of the method. For example, the hard tissue generating growth factor may be added to the complex directly after step a), i.e. directly after mixing of chitosan and negatively charged polysaccharide. In this case, the mixture of carrier and hard tissue generating growth factor is completed prior to shaping of the composition into a solid or semi-solid bone graft substitute structure. Alternatively, the hard tissue generating growth factor may be added during or after setting of the solid or semi-solid bone graft substitute structure, such as in a step directly after step b), during step c), or after step c). For reasons of storage stability of the components of the composition, it may be beneficial to first prepare the solid or semi-solid bone graft substitute structure without addition of hard tissue generating growth factor growth factor, and to subsequently add hard tissue generating growth factor immediately prior to clinical use of the bone graft substitute.

This practice is exemplified by an embodiment of this aspect of the invention, in which the ionic complex is lyophilized to a lyophilizate. The lyophilizate may adopt the form of a dry, sponge-like solid. Shaping of the bone graft substitute may be performed prior to or after lyophilization, as well as prior to or after addition of hard tissue generating growth factor. Such use of a lyophilizate of the inventive composition in the method according to this aspect of the invention, for example in the form of a sponge-like structure, has the added benefit of exploiting the surprisingly advantageous effect of having the composition of ionic complex and hard tissue generating growth factor in the physical form of a lyophilizate.

Steps b) and c) of the method of the invention may advantageously be performed in a mould.

In a third aspect of the invention, there is provided a kit comprising:
a first container containing an ionic complex of i) chitosan and ii) a negatively charged polysaccharide selected from the group consisting of heparin, heparan sulfate and dextran sulfate;
a second container containing a hard tissue generating growth factor; and
instructions to carry out the method according to the second aspect of the invention.

The kit according to this aspect of the invention is suitable for carrying out the inventive method of preparation of a bone graft substitute.

Different embodiments of additional features of the method and/or kit, such as particular choices of hard tissue generating growth factor, negatively charged polysaccharide, chitosan properties etc, are as described above in relation to the composition aspect of the present invention.

In a fourth aspect, the invention provides use of a composition according to the first aspect of the invention in the preparation of a medical device for generation of hard tissue in a mammalian subject in need thereof.

Also provided, in a fifth aspect of the invention, is a method of generation of hard tissue at a desired site in a mammalian subject in need thereof, which method comprises administering to said site of an effective amount of a composition according to the first aspect of the invention under conditions that allow said composition to exert its biological function to generate hard tissue at said site.

In embodiments of these aspects of the invention, the subject may suffer from a condition selected from spinal disc degeneration (necessitating spinal fusion), non-healing long bone fractures (e.g. tibial fractures), bone loss due to surgery (e.g. bone flap necrosis after neurosurgery; bone tumor resection) or trauma (e.g. traffic accidents with crush injuries of the skeleton) and congenital bone defects (e.g. alveolar clefts or other craniofacial deformities).

In other embodiments, the subject may be in need of enhanced osseointegration in connection with an implant, for example dental implants and orthopedic prostheses in the hip, knee or other anatomical regions.

In other embodiments, the subject may be in need of bone reconstruction. In these cases, the inventive composition may be used for purposes of bone prefabrication, e.g. in a muscle or in fat. The prefabricated bone may subsequently be used in bone reconstruction procedures.

All features of all embodiments of all aspects of the invention can be used in any possible combination thereof, provided that such combination is not demonstrably unfeasible as determined without undue experimentation by a person having ordinary skill in the art.

As used herein, the term “hard tissue” is intended to encompass tissue having a firm intercellular substance, and/or tissue that have become mineralized. Examples of hard tissue are bone and cartilage.

In the context of the present invention, the term “bone graft substitute” is intended to encompass all structures that may be used for generation of bone tissue when introduced into a mammalian body and/or brought into contact with mammalian tissue. A bone graft substitute may also, interchangeably, be denoted “bone tissue structure”.

As used herein, the terms “mammals”, “mammalian” etc. include human beings unless otherwise indicated.

### BRIEF DESCRIPTION OF THE DRAWINGS

FIG. 1 is a diagram depicting volume of newly formed bone tissue in rats receiving BMP-2 in the indicated carriers.

FIG. 2 is a diagram depicting density of newly formed bone tissue in rats receiving BMP-2 in the indicated carriers.

### EXAMPLES

For the further understanding of the invention the following non-limiting examples are given:

### Example 1

### Comparison of Different BMP-2 Carriers

### A. Preparation of Collagen/BMP-2 Composition

Bovine type I collagen (Vitrogen 100, Cohesion, Palo Alto, Calif.) was prepared as described by the manufacturer. Briefly, 8 ml chilled Vitrogen collagen was mixed with 1 ml of 10× phosphate-buffered saline solution and 1 ml of 0.1 M NaOH. The pH of the mixture was monitored by pH paper and adjusted to 7.4 through addition of a few drops of 0.1 M HCl or 0.1 M NaOH. The neutralized collagen solution was stored at 4° C. Within 30 minutes, recombinant human BMP-2 (InductOs, Wyeth Lederle) was added by stirring to final concentrations of 50 μg BMP-2 per ml collagen gel or 250 μg BMP-2 per ml collagen gel. The BMP/collagen gels were transferred to 1 ml syringes and kept at room temperature for approximately 10-15 minutes. After that, samples of 0.2 ml were injected into animals as described below.

### B. Preparation of Heparin/Collagen/BMP-2 Composition

The procedure described in A. was repeated, except that BMP-2 was mixed with 1000 IU heparin (sodium heparin, Pharmacia) prior to addition to the collagen gel.

### C. Preparation of Chitosan/BMP-2 Composition

Chitosan (4.5 g) (ChitoClear®, Primex ehf, Norway) with a degree of deacetylation of 84% was added to 125 g water. HCl (4 M) was added drop-wise, at room temperature, to the stirred mixture. The reaction was terminated when a clear solution with a pH of 4.7 had been obtained. The solution was kept over night in a closed vessel. The volume was adjusted to 150 ml and the pH value to 5.0.

To this chitosan solution was added recombinant human BMP-2 (InductOs, Wyeth Lederle) by stirring to final concentrations of 50 μg BMP-2 per ml gel or 250 μg BMP-2 per ml gel. The BMP-chitosan gels were transferred to 1 ml syringes and samples of 0.2 ml were injected into the animals as described below.

### D. Preparation of Chitosan/Heparin/BMP-2 Composition

Chitosan (4.5 g) (ChitoClear®, Primex ehf, Norway) with a degree of deacetylation of 84% was added to 125 g water. HCl (4 M) was added drop-wise, at room temperature, to the stirred mixture. The reaction was terminated when a clear solution with a pH of 4.7 had been obtained. Heparin (1.8 g) (sodium heparin, Pharmacia) was dissolved in 25 g of water. The solution was kept over night in a closed vessel. The heparin solution was added to the chitosan solution under stirring. The weight ratio of chitosan:heparin in the final preparation was 5:2 and the pH of the gel formed was 5.7. After adjusting the pH-value to 4.9 with 4 M HCl, water was added to a final weight of the preparation of 150 g. Examination of the resulting gel showed that it was macroscopically homogenous with no phase separations. Stability was good.

To the chitosan/heparin gel was added recombinant human BMP-2 (InductOs, Wyeth Lederle) by stirring to final concentrations of 50 μg BMP-2 per ml gel or 250 μg BMP-2 per ml gel. The chitosan/heparin/BMP-2 gels were transferred to 1 ml syringes and samples of 0.2 ml were injected into the animals as described below. Each injection contained 6.0 mg chitosan and 2.40 mg heparin together with BMP-2.

### E. Effect of Different BMP-2 Compositions on Bone Formation

Sixty adolescent male Sprague Dawley rats weighing 250-300 grams were anaesthesized with Temgesic® (0.16 ml/kg body weight). Into each rat, 0.2 ml of one of the four different BMP-2 gels prepared as described above was injected into the quadriceps muscle using a 22 G needle. The rats were divided into twelve groups of five rats in each group. Each rat received 0 μg BMP-2, 10 μg BMP-2 or 50 μg BMP-2 in one of the four different carriers collagen, heparin/collagen, chitosan and heparin/chitosan. The animals were allowed to move freely after the procedure. The animals were euthanasized by CO₂ at four weeks post-injection.

### F. Radiography and CT Scan

Four weeks after the injections, the hind legs were cut off from euthanasized animals and radiography was performed. Small animal CT scans were performed on the same hind legs to calculate induced ectopic bone volume and bone mineral density.

### G. Results and Discussion

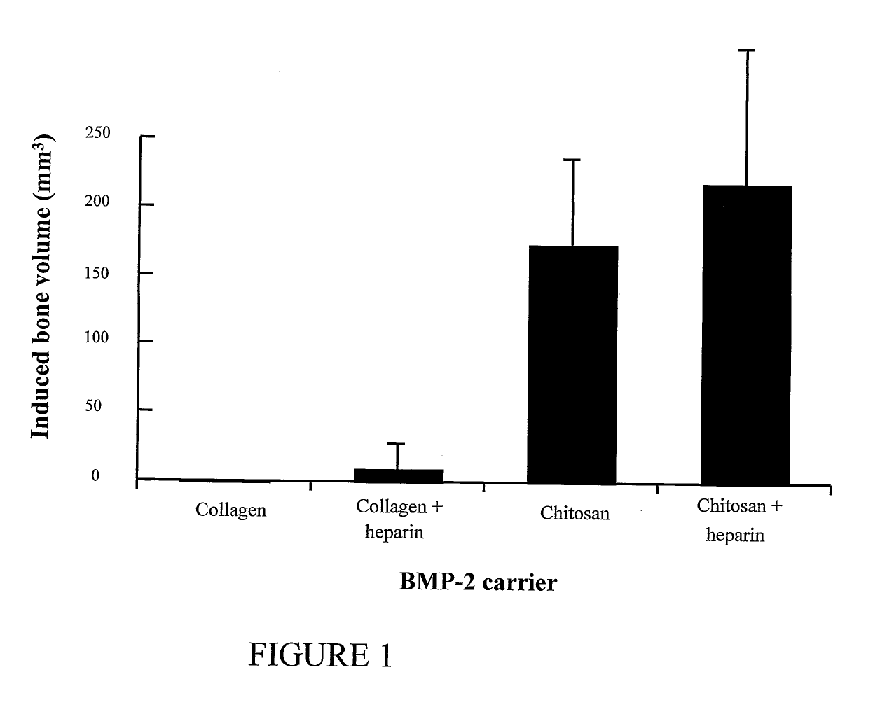

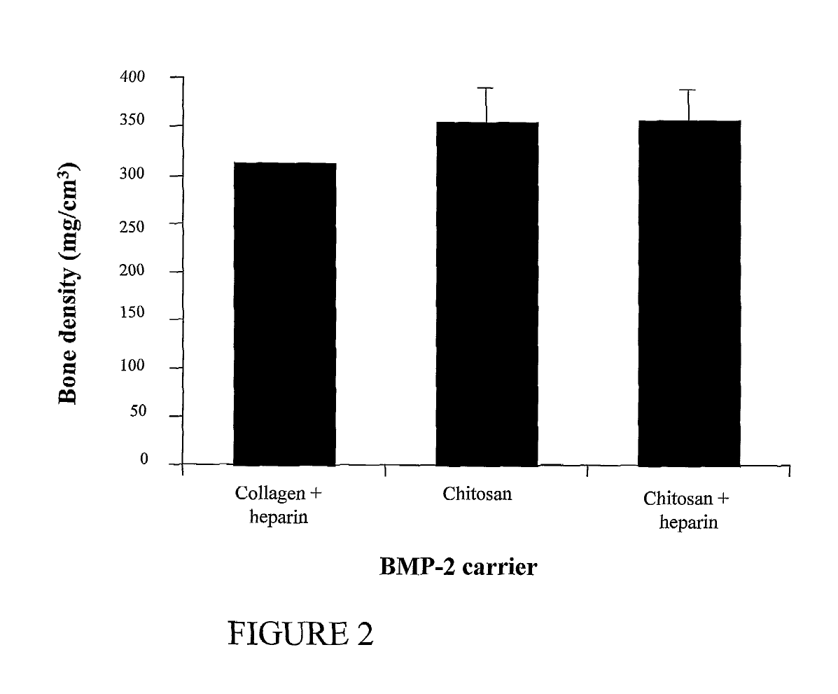

The results with regard to both the volume and density of the newly formed bone tissue are presented in Table 1 and in FIGS. 1 and 2.

TABLE 1

| | BMP-2 | Bone volume | Bone density |
|---|---|---|---|
| BMP-2 carrier | (μg) | (mm³) | (mg/cm³) |
| Collagen (A) | 0 | 0 | 0 |
| | 10 | 0 | 0 |
| | 50 | 0 | 0 |
| Collagen/heparin (B) | 0 | 0 | 0 |
| | 10 | 0 | 0 |
| | 50 | 9 | 313 |
| Chitosan (C) | 0 | — | — |
| | 10 | — | — |
| | 50 | 173 | 355 |
| Chitosan/heparin (D) | 0 | 0 | 0 |
| | 10 | 31 | 351 |
| | 50 | 218 | 356 |

No bleeding complications or thrombus formation was observed in the experiments involving collagen only or the combination of chitosan and heparin.

Collagen and collagen/heparin without BMP-2 did not induce any bone formation, whereas the chitosan/heparin complex alone induced a small amount of ectopic bone in one specimen out of five. Neither collagen or collagen/heparin induced ectopic bone formation when mixed with 10 μg BMP-2. Limited bone formation was induced by chitosan/heparin with 10 μg BMP-2. When increasing the amount of BMP-2 to 50 μg, the collagen/heparin carrier induced bone formation in one specimen. The amounts of ectopic bone formation were dramatically increased in all specimens when chitosan alone was used as delivery vehicle together with 50 μg BMP-2. The effect on induced bone volume was even higher when chitosan/heparin was used as BMP-2 carrier (Table 1, FIG. 1). No significant difference in bone mineral density was calculated between the groups (FIG. 2).

The experiment with 50 μg BMP-2 was repeated, yielding almost identical results.

### Example 2

### Lyophilized BMP-2 carriers

### A. Preparation of Lyophilized Chitosan/Heparin Complex

Chitosan (9.0 g) (ChitoClear®, Primex ehf, Norway) was, under stirring, added to approximately 200 g pure water. HCl (4 M, aq) was added drop-wise under stirring. When the chitosan had been completely dissolved, pH was adjusted to 4.7±0.2. The solution was left at room temperature for one hour, and water was added to a final volume of 246.4 ml. An aqueous solution of heparin (3.6 g in 50 ml) (sodium heparin, Pharmacia) was added, under stirring, to the chitosan solution during 5 minutes. The pH-value of the resulting gel was 5.0.

The thus formed heparin/chitosan complex gel was poured into Petri dishes with a diameter of 9 cm, in an amount of 40 g of complex gel per dish. The dishes were placed in a freezer at approximately −10° C. until the complex had been completely frozen. The Petri dishes were transferred to a freeze-dryer and dried until complete dryness, which resulted in a white, sponge-like solid material of the complex.

### B. Preparation and Implantation of Lyophilized Carrier/BMP-2 Compositions

Heparin/chitosan sponges as described above and type I collagen sponges (InductOs, Wyeth Lederle) were cut into 4×6 mm pieces. 50 μg BMP-2 in aqueous solution (InductOs, Wyeth Lederle) was added to each group of sponges. The 4×6 mm heparin/chitosan sponges contained 6.1 mg chitosan and 2.42 mg heparin each. The sponges with adsorbed BMP-2 were kept at room temperature for at least 15 minutes and were then implanted into the quadriceps muscles in adolescent male Sprague Dawley rats through a 15 mm skin incision. Six implantations were performed of each of the two different materials. The animals were allowed to move freely after the procedure.

### C. Results and Discussion

Ectopic bone formation was studied by x-ray 4 weeks post-implantation. Abundant bone formation was observed in 5 out of 6 rats in the chitosan/heparin/BMP group, whereas no bone formation was observed in the collagen/BMP group.

The effect on induced bone when the lyophilized “heparin/chitosan sponge” was used as BMP-2 delivery vehicle was superior to that observed in Example 1 with the ionic complex of heparin and chitosan in the form of a gel, in that the bone formation was restricted to a clearly delimited area defined by the lyophilizate implant.

### Example 3

### Comparison of different BMP-4 carriers

Examples 1 and 2 are repeated, except that BMP-4 is used instead of BMP-2.

Similar results with regard to bone formation are expected.

### Example 4

### Case Study

### Background

A 42-year-old woman was referred to the clinic with a large cranial bone defect. Four years previously, she had been treated for a brain tumor by surgery and postoperative irradiation. A bone flap measuring 7×9 cm in her right fronto-parietal region had been lost due to an infection following surgery. More than four attempts of cranial reconstruction using different implants had failed because of infections and penetration of the implants through the skin.

### Procedure

Preoperative photographs of the patient's cranial defect revealed that it was located at the right fronto-parietal area of the skull.

12 mg BMP-2 (as provided in the InductOs kit from Wyeth; a powder containing recombinant human BMP-2, saccharose, glycine, glutamic acid, NaCl, polysorbat 80 and NaOH) was dissolved in 8 ml sterile water (InductOs kit, Wyeth). The BMP solution was dispersed onto two samples of sterile sponges of chitosan-heparin lyophilizate (7×9×0.5 cm) prepared as described in Example 2.

The defect was surgically exposed, and one of the sponges was applied directly onto the dura, followed by a cranial titanium mesh (Walter Lorenz Surgical Inc., Jacksonville, Fla., USA) which served as a scaffold. The titanium mesh was covered with the second chitosan-heparin sponge to remodel the cranial contour.

Finally, the implants were covered by a local periosteal flap before skin closure.

### Results

Bacterial testing before surgery demonstrated that the chitosan-heparin sponges were sterile.

Postoperative CT-scan demonstrated calcified dura at the implantation site.

No bleeding complications were observed and the APTT levels were normal throughout the treatment and afterwards. After 6-8 weeks, hair loss occurred at the implantation site, which is a well-known side effect in connection with treatment with BMP-2. Hair re-grew after approximately 16-20 weeks.

After 8 months, the patient has had no infections or penetrations of the titanium mesh, despite the fact that these are common complications in reconstruction and occurred on all previous attempts to treat the patient.

1. Composition capable of generating hard tissue when introduced in a mammalian subject, the composition comprising:
a) an ionic complex of i) chitosan and ii) a negatively charged polysaccharide selected from the group consisting of heparin, heparan sulfate and dextran sulfate, and
b) a hard tissue generating growth factor,
said ionic complex being a carrier for said hard tissue generating growth factor.

2. Composition according to claim 1, in which the number of positive charges contributed by said chitosan are in excess over the number of negative charges contributed by said negatively charged polysaccharide, in said ionic complex.

3. Composition according to claim 1, in which said hard tissue generating growth factor is selected from the group consisting of BMP-2, BMP-4, BMP-6, BMP-7, BMP-9 and BMP-14.

4. Composition according to claim 3, in which said hard tissue generating growth factor is BMP-2.

5. Composition according to claim 1 in which said negatively charged polysaccharide is heparin.

6. Composition according to claim 5, in which the weight ratio chitosan:heparin is from about 1:2 to about 10:1.

7. Composition according to claim 6, in which the weight ratio chitosan:heparin is from about 1:1 to about 5:1.

8. Composition according to claim 7, in which the weight ratio chitosan:heparin is from about 2:1 to about 5:1.

9. Composition according to claim 8, in which the weight ratio chitosan:heparin is from about 3:1 to about 4:1.

10. Composition according to claim 8, in which the weight ratio chitosan:heparin is from about 2:1 to about 3:1.

11. Composition according to claim 1, in which said chitosan has a degree of deacetylation of from about 50% to about 98%.

12. Composition according to claim 11, in which said chitosan has a degree of deacetylation of from about 80% to about 90%.

13. Composition according to claim 1, in which the content of said hard tissue generating growth factor is from about 0.1 to about 10 percent by weight, preferably from about 0.5 to about 5 percent by weight, based on the total weight of ionic complex and hard tissue generating growth factor.

14. Composition according to claim 1, in which said ionic complex is in the form of a gel.

15. Composition according to claim 14, in which said hard tissue generating growth factor is present in a concentration of from about 5 to about 500 μg/ml gel, preferably from about 1 to about 100 μg/ml gel.

16. Composition according to claim 1, in which said ionic complex is in the form of a lyophilizate.

17. Composition according to claim 16, in which said hard tissue generating growth factor is present in a concentration of from about 1 to about 50 μg/mg lyophilizate, preferably from about 2 to about 25 μg/mg lyophilizate.

18. Method of preparation in vitro of a bone graft substitute, which method comprises:
a) providing an ionic complex of i) chitosan and ii) a negatively charged polysaccharide selected from the group consisting of heparin, heparan sulfate and dextran sulfate,
b) shaping said ionic complex in a desired shape of a bone graft substitute, and
c) allowing said ionic complex to set into a solid or semi-solid bone graft substitute structure with said desired shape,
which method also comprises the step of adding a hard tissue generating growth factor to said ionic complex.

19. Method according to claim 18, wherein steps b) and c) are performed in a mould.

20. Method according to claim 18, which further comprises lyophilization of said ionic complex to a lyophilizate.

21. Method according to claim 20, in which the step of adding a hard tissue generating growth factor comprises adsorption of a solution of said growth factor onto said lyophilizate.

22. Method according to claim 18, in which said hard tissue generating growth factor is selected from the group consisting of BMP-2, BMP-4, BMP-6, BMP-7, BMP-9 and BMP-14.

23. Method according to claim 18, in which said negatively charged polysaccharide is as heparin.

24. Method according to claim 18, in which said chitosan is has a degree of deacetylation of from about 50% to about 98%.

25. Method according to claim 18, in which the number of positive charges contributed by said chitosan are in excess over the number of negative charges contributed by said negatively charged polysaccharide, in said ionic complex.

26. Kit comprising
a first container containing an ionic complex of i) chitosan and ii) a negatively charged polysaccharide selected from the group consisting of heparin, heparan sulfate and dextran sulfate;
a second container containing a hard tissue generating growth factor; and
instructions to carry out the method according to claim 18.

27. Kit according to claim 26, in which said hard tissue generating growth factor is selected from the group consisting of BMP-2, BMP-4, BMP-6, BMP-7, BMP-9 and BMP-14.

28. Kit according to claim 26, in which said negatively charged polysaccharide is heparin.

29. Kit according to claim 26, in which said chitosan has a degree of deacetylation of from about 50% to about 98%.

30. Kit according to claim 26, in which the number of positive charges contributed by said chitosan are in excess over the number of negative charges contributed by said negatively charged polysaccharide, in said ionic complex.

31. Use of a composition according to claim 1 in the preparation of a medical device for generation of hard tissue in a mammalian subject in need thereof.

32. Use according to claim 31, in which said hard tissue is bone tissue.

33. Use according to claim 31, in which said subject suffers from a condition selected from spinal disc degeneration, non-healing long bone fractures, bone loss due to surgery or trauma and congenital bone defects.

34. Use according to claim 31, in which said subject is in need of enhanced osseointegration in connection with an implant.

35. Use according to claim 31, in which said subject is in need of bone reconstruction.

36. Method of generation of hard tissue at a desired site in a mammalian subject in need thereof, which method comprises administering to said site of an effective amount of a composition according to claim 1 under conditions that allow said composition to exert its biological function to generate hard tissue at said site.

37. Method according to claim 36, in which said hard tissue is bone tissue.

38. Method according to claim 36, in which said subject suffers from a condition selected from spinal disc degeneration, non-healing long bone fractures, bone loss due to surgery or trauma and congenital bone defects.

39. Method according to claim 36, in which said subject is in need of enhanced osseointegration in connection with an implant.

40. Method according to claim 36, in which said subject is in need of bone reconstruction.
